(19) United States
(12) Patent Application Publication
(10) Pub. No.: US 2015/0282972 A1
(43) Pub. Date: Oct. 8, 2015

(54) MULTIFUNCTIONAL MASSAGE JOINT PROTECTOR

(71) Applicant: Mei-Hui Tsai, Taichung City (TW)

(72) Inventor: Mei-Hui Tsai, Taichung City (TW)

(73) Assignee: FUNCARE OF TAIWAN CO., LTD., Taichung City, Taiwan (CN)

(21) Appl. No.: 14/441,780

(22) PCT Filed: Dec. 19, 2012
(86) PCT No.: PCT/CN2012/086920

§ 371 Date: May 8, 2015

(51) Int. Cl. A61F 5/01; A61H 1/00

(52) U.S. Cl. CPC A61F 5/0104; A61H 1/008

(57) ABSTRACT

A multifunctional massage joint protector, comprising an elastic piece body, a palm-shaped and a low-frequency massage part. The elastic piece body is used for coating and fixed on a human joint, the palm-shaped coating part coats the periphery of the human joint, and the low-frequency massage part is adjacent to the palm-shaped coating part and arranged on the elastic piece body. The joint protector, on one hand, uses the palm shape of the palm-shaped coating part to locate the human joint, in order to avoid the problem of shifting of the protector due to detachment when the human joint moves, and uses the low-frequency massage part to enhance the effect of the kneading and massaging, and on the other hand, coats the human joint by means of the palm-shaped coating part and the massage possesses a tactile sensation, thereby improving the use value of the protector.

### FIELD OF THE INVENTION

The present invention relates to a protector and particularly to a multifunctional massage joint protector

### BACKGROUND OF THE INVENTION

To avoid human body from injury many types of protective means to protect various body portions have been designed and available at present. Among the injuries that might happen to human body injury of joints often is very difficult to heal. Hence providing proper protection of the joints is very important. In addition, in the event that people have suffered injury on a portion of the body or osteoporosis, a protector also is required to provide proper protection. The conventional protector often provides a resilient and pliable wrapping material of a large area to wrap the joint to achieve protection effect. But when people move, such as walking, the movement of the joint easily dislocates the protector from its original intended position, hence makes joint protection less effective.

Hence how to make a protector that can be snugly attached to the joint without dislocating is an issue yet to be satisfactorily resolved in the therapeutic field.

### SUMMARY OF THE INVENTION

The primary object of the present invention is to solve the problem of the conventional protectors of unable to form snug attachment to the human joint that results in dislocation of the protectors and deficiency in protection.

To achieve the foregoing object the present invention provides a multifunctional massage joint protector to wrap a human joint to provide protection. It includes an elastic piece body, a palm-shaped coating part and a low-frequency massage part. The elastic piece body includes a first connecting portion, a second connecting portion and a main body located between the first connecting portion and the second connecting portion. The palm-shaped coating part is located at one side of the main body and includes a first palm-shaped zone, a first finger-shaped zone and a first recess located on the first palm-shaped zone. The first recess corresponds to the human joint to allow the first palm-shaped zone to wrap the periphery of the human joint. The first connecting portion and the second connecting portion are connected to each other to allow the elastic piece body to wrap the human joint with the palm-shaped coating part abutting one side of the human joint. The low-frequency massage part is abutting the palm-shaped coating part and located on the main body to massage the human joint through low frequency waves.

Through the structure set forth above, the invention provides many advantageous features, notably:

1. With the first recess of the palm-shaped coating part the human joint can be securely positioned on the first palm-shaped zone without dislocating during movement of the human joint, thus can resolve the problem of protection deficiency caused by dislocation of the protector during joint movement that might otherwise occur.

2. By forming snug attachment of the palm-shaped coating part to the human joint to provide a chiropractic-like tactility, a muscle soothing and massaging efficacy can be achieved to increase the added value of the protector.

3. Through the low-frequency massage part blood circulation can be enhanced, and nerve and vital points can be stimulated to eliminate fatigue, stiffness and sore of human body.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a front perspective view of the invention.

FIG. 2 is a rear perspective view of the invention.

FIG. 3 is a schematic view of the invention in a use condition.

FIG. 4 is a rear perspective view of another embodiment of the invention.

### DETAILED DESCRIPTION OF THE PREFERRED EMBODIMENTS

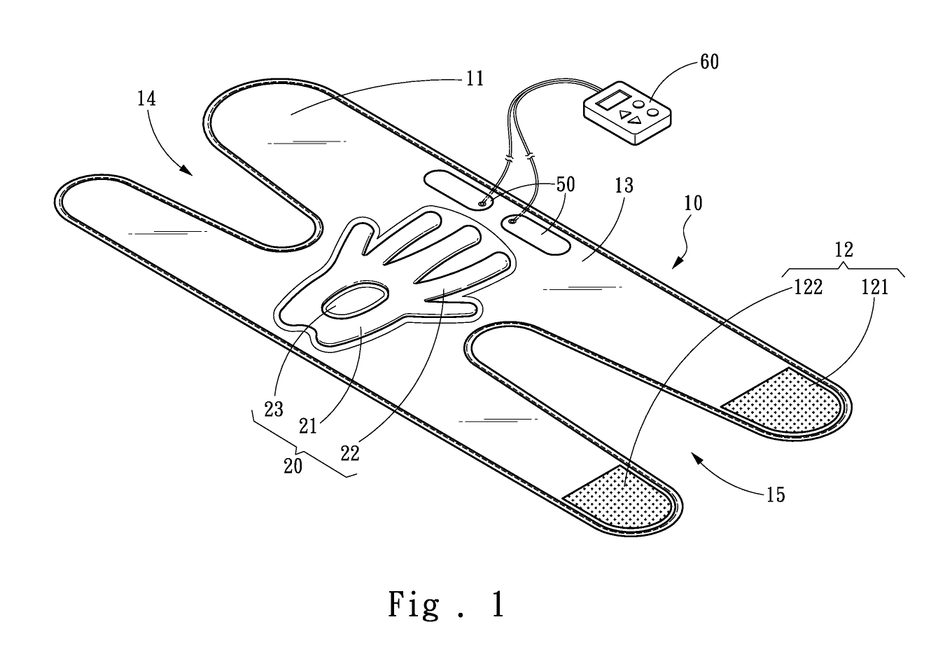

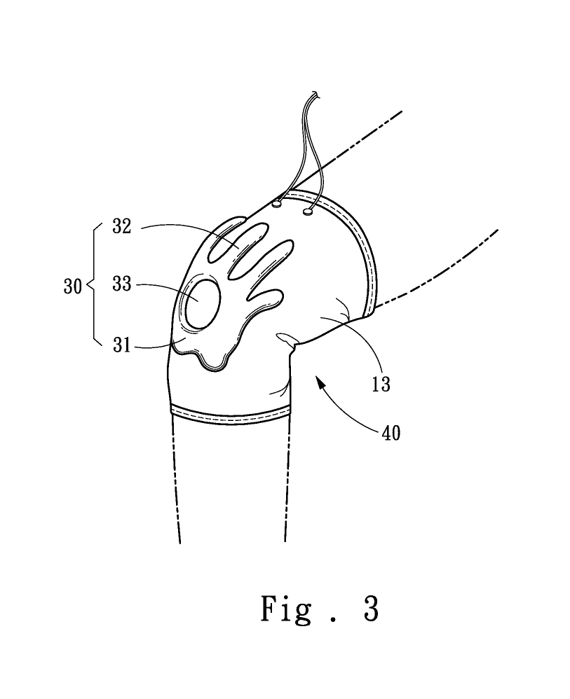

Please referring to FIGS. 1, 2 and 3, the present invention aims to provide a multifunctional massage joint protector to wrap a human joint 40 (referring to FIG. 3) to provide protection. It includes an elastic piece body 10, a palm-shaped coating part 20 and a low-frequency massage part 50. The elastic piece body 10 includes a first connecting portion 11, a second connecting portion 12 and a main body 13 located between the first connecting portion 11 and the second connecting portion 12. The palm-shaped coating part 20 is located at one side of the main body 13 and includes a first palm-shaped zone 21, a first finger-shaped zone 22 and a first recess 23 located on the first palm-shaped zone 21. The first finger-shaped zone 22 has a plurality of finger-shaped bodies. In this embodiment five finger-shaped bodies are provided to mate human fingers as an example. The first recess 23 corresponds to the human joint 40 to allow the first palm-shaped zone 21 to wrap the periphery of the human joint 40. The first connecting portion 11 and the second connecting portion 12 are connected to each other to allow the elastic piece body 10 to wrap the human joint 40. The palm-shaped coating part 20 is abutting one side of the human joint 40. In this embodiment the first connecting portion 11 and the second connecting portion 12 are Velcro bands. The low-frequency massage part 50 is abutting the palm-shaped coating part 20 and located on the main body 13. In this embodiment the low-frequency massage part 50 is adjacent to one end of the first finger-shaped zone 22 remote from the first palm-shaped zone 21. The low-frequency massage part 50 can be designed and located at other positions according to use requirements. In addition, the low-frequency massage part 50 is electrically connected to a power output control unit 60 to do control thereof, such as ON/OFF, strong and weak, and massage types.

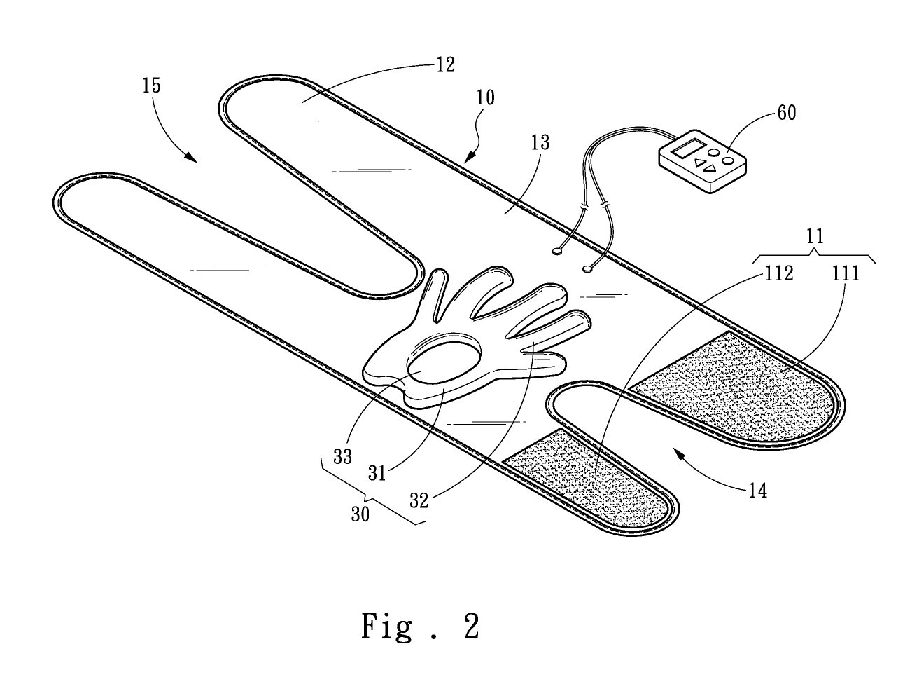

More specifically, referring to FIGS. 2 and 3, the invention further includes an outer palm-shaped portion 30 corresponding to the palm-shaped coating part 20 that includes a second palm-shaped zone 31, a second finger-shaped zone 32 and a second recess 33 located on the second palm-shaped zone 31. The second finger-shaped zone 32 mates the first finger-shaped zone 22 with a plurality of finger-shaped bodies. The second recess 33 corresponds to the human joint 40, also referring to FIG. 1. The outer palm-shaped portion 30 is formed at a thickness greater than that of the palm-shaped coating part 20. Furthermore, the first connecting portion 11 has a first slit zone 14 to form a first upper connecting sheet 111 and a first lower connecting sheet 112, and the second connecting portion 12 has a second slit zone 15 to form a second upper connecting sheet 121 and a second lower connecting sheet 122. Through the first slit zone 14 and the second slit zone 15, the protector of invention can snugly fit the human joint 40 for movement when in use such as bending or stretching without affecting motion of the human joint 40 caused by connection of the first connecting portion 11 and the second connecting portion 12.

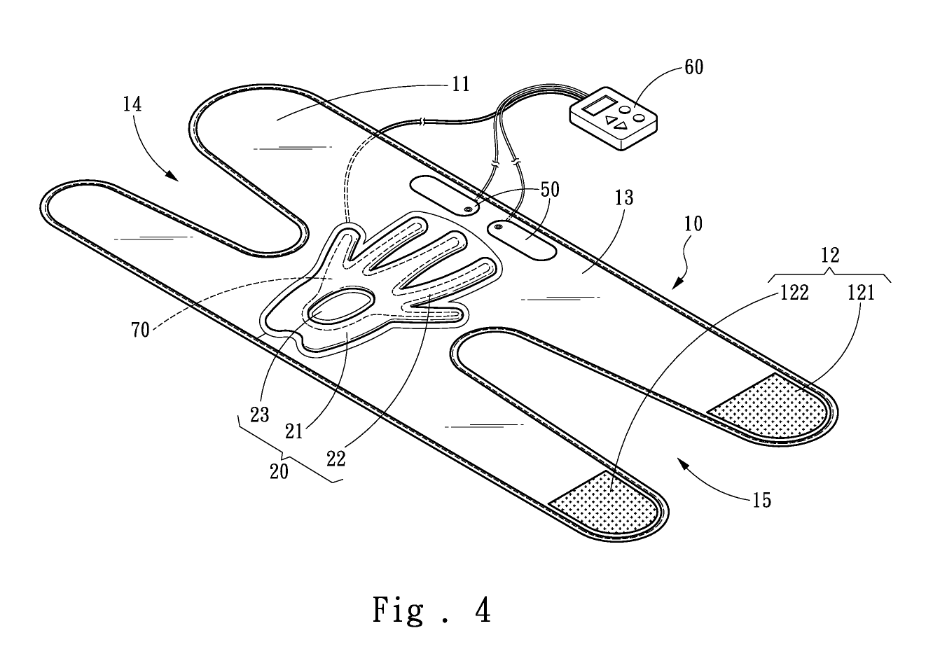

Please refer to FIG. 4 for another embodiment of the invention. The invention further has a fomentation portion 70 located on the palm-shaped coating part 20 that is electrically connected to the power output control unit 60 to achieve fomentation effect.

As a conclusion, the invention provides many advantageous features, notably:

1. With the first recess formed on the palm-shaped coating part the human joint can be securely positioned in the first palm-shaped zone without dislocating during movement of the human joint, thus resolve the problem of protection deficiency that might otherwise occur.

2. By forming snug attachment of the palm-shaped coating part to the human joint to provide chiropractic-like tactility, a muscle soothing and massaging efficacy can be achieved to increase the added value of the protector.

3. Through the outer palm-shaped portion improved protection effect can be provided.

4. Through the low-frequency massage part blood circulation can be enhanced, and nerve and vital points can be stimulated to eliminate fatigue, stiffness and sore of human body.

5. Through the fomentation portion can be provided to alleviate muscle stiffness and soreness.

In short, the present invention can provide significant improvements over the conventional techniques.

While the preferred embodiments of the invention have been set forth for the purpose of disclosure, they are not the limitation of the invention, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A multifunctional massage joint protector to wrap a human joint to provide protection, comprising:
an elastic piece body including a first connecting portion, a second connecting portion and a main body located between the first connecting portion and the second connecting portion;
a palm-shaped coating part which is located at one side of the main body and includes a first palm-shaped zone, a first finger-shaped zone and a first recess located on the first palm-shaped zone corresponding to the human joint to allow the first palm-shaped zone to wrap the periphery of the human joint; and
a low-frequency massage part located on the main body and abutting the palm-shaped coating part;
wherein the first connecting portion and the second connecting portion are connected to each other to allow the elastic piece body to wrap the human joint with the palm-shaped coating part abutting one side of the human joint.

2. The multifunctional massage joint protector of claim 1 further including an outer palm-shaped portion corresponding to the palm-shaped coating part and including a second palm-shaped zone, a second finger-shaped zone and a second recess located on the second palm-shaped zone that corresponds to the human joint.

3. The multifunctional massage joint protector of claim 2, wherein the outer palm-shaped portion is formed at a thickness greater than that of the palm-shaped coating part.

4. The multifunctional massage joint protector of claim 1, wherein the first connecting portion includes a first slit zone to form a first upper connecting sheet and a first lower connecting sheet thereof, and the second connecting portion includes a second slit zone to form a second upper connecting sheet and a second lower connecting sheet thereof.

5. The multifunctional massage joint protector of claim 1, wherein the first connecting portion and the second connecting portion are Velcro bands.

6. The multifunctional massage joint protector of claim 2, wherein the first finger-shaped zone and the second finger-shaped zone include respectively a plurality of finger-shaped bodies that correspond to each other.

7. The multifunctional massage joint protector of claim 1 further including a power output control unit which is electrically connected to the low-frequency massage part.

8. The multifunctional massage joint protector of claim 7 further including a fomentation portion located on the palm-shaped coating part that is electrically connected to the power output control unit.

9. The multifunctional massage joint protector of claim 1, wherein the low-frequency massage part is located at one end of the first finger-shaped zone remote from the first palm-shaped zone.
